(19) United States
(12) Patent Application Publication
(10) Pub. No.: US 2003/0170199 A1
(43) Pub. Date: Sep. 11, 2003

(54) Cosmetic and/or dermatological composition based on cocoa extracts

(76) Inventor: Jacques Leclere, Saint-Gondon (FR)

Correspondence Address: BURNS DOANE SWECKER & MATHIS L L P, POST OFFICE BOX 1404, ALEXANDRIA, VA, 22313-1404, US

(21) Appl. No.: 10/311,544

(22) PCT Filed: Jun. 15, 2001
(86) PCT No.: PCT/FR01/01885

(30) Foreign Application Priority Data
Jun. 16, 2000 (FR) 00/07748

(51) Int. Cl. A61K 7/06; A61K 35/78

(52) U.S. Cl. 424/74; 424/776

(57) ABSTRACT

The invention relates to a cosmetic and/or dermatological composition.

The cosmetic and/or dermatological composition essentially comprises an extract of cocoa containing polyphenols, and more particularly an extract of cocoa containing, in combination, polyphenols, amino acids and a concentrate of unsaponifiable materials.

Application to topical-route compositions in the care and prevention of the signs of ageing of the skin.

The present invention relates to a novel cosmetic and/or dermatological composition, and more particularly to a composition based on extracts of cocoa (Theobroma cacao) with excellent properties that may be used in cosmetics and dermatology to care for and protect the skin.

The skin comprises surface layers, namely the epidermis, and deeper layers, the dermis and the hypodermis, and each one has specific properties allowing the ensemble to react and to adapt to the conditions of its environment. The epidermis, which constitutes the outer layer, plays a fundamental role in providing protection and maintaining good trophicity. Consequently, many compositions have been developed in order to protect it and to improve its functions.

These cosmetic and dermatological compositions intended for treating skin complaints by topical application must satisfy certain conditions in order to be accepted by users. More particularly, they must have good physical properties, especially in terms of consistency and creaminess, they must have satisfactory efficacy and they must be pleasant to use. Furthermore, they must be able to be stored under normal temperature and hygrometry conditions without substantially degrading and while maintaining their properties over prolonged periods.

Besides these physical properties, the compositions must have a combination of activities that may be used cosmetically and pharmaceutically. In particular, the compositions generally sought are those combining properties of moisturizing and protecting the skin against environmental attack or ageing, manifested by the formation of wrinkles and marks, greater dryness and loss of elasticity of the skin.

Free radicals are known for their effect of accelerating cellular ageing. Their formation is often promoted by external factors such as ultraviolet rays, pollution and the action of certain medicinal products. The systems for protecting the body against free radicals have a tendency to lose their efficacy with age and it is thus desirable to have available substances capable of neutralizing the harmful effects of free radicals. Various polyphenols are frequently used for this purpose. For example, patent FR-A-2 749 303 describes a process for extracting catechol polyphenols from plants using an organic solvent and their use in cosmetic compositions with free-radical-scavenging properties that are useful against ageing of the skin.

It is known that cocoa has stimulating, tonic, nutrient and stress-relieving effects and various substances have been extracted from cocoa on account of these properties. In particular, cocoa butter is used in many food, cosmetic and pharmaceutical compositions. The cacao tree (Theobroma cacao) is a shrub originating from the forests of Central America and the equatorial forests of South America. Various substances have been extracted from the beans or shells. A process for treating cocoa beans to obtain dietetic and food products containing polyphenols is described in patent U.S. Pat. No. 6,015,913. Patent JP-A-00 128 801 describes an antibacterial composition comprising an extract of mint and an extract of tea leaves or of cocoa beans containing polyphenols, which may be used in food products and drinks. Patent FR-A-2 413 042 describes a process for extracting cocoa shells using an acidic solution of an alcohol, and the use of the product thus obtained in food compositions. However, it has not yet been envisaged to use a total extract of cocoa in cosmetic or dermatological compositions.

One subject of the present invention is a novel composition that may be used in cosmetics and/or in dermatology, obtained by extraction of cocoa, and containing polyphenols.

A subject of the invention is also a cosmetic and/or dermatological composition based on a protein extract of cocoa (Theobroma cacao) comprising at least polyphenols, amino acids and an unsaponifiable fraction.

A subject of the invention is also the use of a composition as indicated above for treating and preventing the signs of ageing of the skin, such as the appearance of wrinkles and the loss of elasticity of the skin.

Finally, a subject of the present invention is also a cosmetological process for treating the skin, which consists in applying to the skin a composition as indicated above.

The composition in accordance with the present invention essentially comprises an extract of cocoa containing polyphenols. More particularly, the composition of the invention comprises an extract of cocoa containing, in combination, polyphenols, amino acids and/or a concentrate of unsaponifiable materials.

According to one preferred embodiment of the invention, the composition contains, in combination:

from 0.05% to 5% by weight of amino acids extracted from cocoa;

from 0.05% to 5% by weight of polyphenols extracted from cocoa;

from 0.05% to 3% by weight of a concentrate of unsaponifiable materials.

The extraction process used in the invention consists in performing an extraction of a known type, for example by grinding cocoa beans and hydrophilic/lipophilic separation of the cocoa butter, on the one hand, and of a mixture of proteins and polyphenols, on the other hand. The hydrophilic/lipophilic separation makes it possible, by extraction of the ground seed, to separate with water the supernatant fatty substances from the polyphenol/protein phase. Water brought to a temperature of between 80 and 100° C. approximately is preferably used.

The proteins are separated out, for example, by tangential ultrafiltration (proteins>10 000 Da), while the polyphenols are dried and then encapsulated, if necessary. The fraction enriched in unsaponifiable materials is obtained from the cocoa butter by molecular distillation at 200-300° C. under vacuum (about 10⁻⁴ to 10⁻² mmHg/0.13 to 13 Pa).

The polyphenol extract thus obtained is in the form of a dark-colored liquid that is soluble in water and alcohols.

The extraction is preferably performed from beans of the cocoa tree (Theobroma cacao). It is thus possible to obtain extracts with a high protein content (greater than 5%) with excellent properties. In particular, the amino acid analysis of the protein extract revealed the following main amino acids. In the table that follows, the amino acid contents are given in grams per 100 g of extract.

| | |
|---|---|
| Glycine | 26.3 |
| Alanine | 18.3 |
| Serine | 14.1 |
| Tyrosine | 9.7 |
| Aspartic acid and asparagine | 4.2 |
| Valine | 3.1 |
| Glutamic acid and glutamine | 2.8 |
| Threonine | 2.0 |
| Arginine | 1.5 |
| Phenylalanine | 1.4 |
| Leucine | 1.1 |
| Isoleucine | 1.1 |
| Lysine | 1.0 |

The analysis also shows the presence, in a content of less than 1% by weight, of proline, cystine, methionine and histidine.

The polyphenol extract of cocoa used in the present invention is in the form of an amber-colored liquid that is soluble in water and alcohols, which has excellent free-radical-scavenging properties, giving the compositions according to the invention good efficacy in the prevention of ageing of the skin and the attenuation of wrinkles.

The polyphenols extracted from cocoa used in the invention are preferably encapsulated, for example in the form of lecithin-based liposomes, the mean size of which is preferably in the region of 100 μm, in order to protect them and to prevent them from polymerizing.

As indicated above, the extract according to the invention also contains a concentrate of unsaponifiable materials which may be separated from the total extract by extraction with carbon dioxide or, preferably, by molecular distillation. This concentrate of unsaponifiable materials contains hydrocarbons, tocopherol, terpene alcohols and sterols, and it has fibroblast-stimulating properties that are particularly advantageous for the dermatological compositions of the invention.

Besides the components of the cocoa extract indicated above, the composition in accordance with the present invention may advantageously contain a hydrogenated lecithin.

The hydrogenated lecithin used in the invention is preferably a hydrogenated soybean lecithin or a hydrogenated sunflower lecithin. The lecithins that are commercially available generally contain pure lecithin (phosphatidyl choline) mixed with other phosphoglycerides such as cephalin (in particular phosphatidyl ethanolamine) and phosphatidylinositol. In accordance with the invention, hydrogenated pure lecithin or hydrogenated commercial lecithin may be used. An example of a hydrogenated lecithin that may be used in the invention is the product marketed under the brand name Emulmetik 320® by the company Lucas Mayer (hydrogenated soybean lecithin).

The compositions may also advantageously contain cocoa oil and cocoa butter.

According to the standard terminology, “topical administration” denotes any method consisting in applying the substance or composition directly to the skin.

In accordance with the present invention, the composition may be administered topically and it may advantageously contain, besides the basic components described above, one or more other substances known to exert complementary beneficial effects on the skin, and more particularly tocopherol, vitamin A (retinol), retinoic acid, bactericides, etc.

The cosmetic or pharmaceutical compositions in accordance with the present invention are intended for topical administration and contain supports and excipients commonly used in compositions of this type, such as O/W or W/O emulsions, creams, gels or lotions. In the case of emulsions, the fatty phase may represent between 10% and 60% approximately of the weight of the composition, the aqueous phase between 10% and 80% approximately and the emulsifier between 2% and 20%, the remainder consisting of the basic components indicated above and the other components mentioned below.

The composition may also contain various substances and excipients chosen as a function of their known properties and of the intended presentation form. Thus, preserving agents, emulsifiers, viscosity enhancers, thickeners, gelling agents, antioxidants, moisturizers, surfactants, fragrances, oils, lipids, a specific solvent and also water and various additives for improving the physical properties of the composition may be incorporated into the composition. Sunscreens or sunblocks chosen as a function of the desired degree of protection may also advantageously be incorporated.

The emulsifier may be chosen from carboxyvinyl polymers of high molecular weight (for example Carbopol®), polysorbates (for example Tween 20® or Tween 60°), sorbitan esters and in particular a sorbitan monostearate, tristearate, monopalmitate or laurate (for example Arlacel®). Other emulsifiers such as various stearic acid or palmitic acid derivatives, for example PEG-100 stearate®, stearic acid or palmitic acid mono- or diglycerides, a self-emulsifying propylene glycol stearate, or polyglyceryl 2-sesquioleate, polyoxyethylene cetyl ether, a siloxane polyglucoside or an emulsifiable silicone may also be used. Mixtures of nonionic emulsifiers such as Protegin X® may also be used.

The viscosity enhancers used in the compositions of the invention may be chosen from various acrylic acid polymers, a cellulose gum, a silica, carboxyvinyl polymers, for example Carbomer®, or a magnesium aluminum silicate, and the colloidal silica sold under the brand name Aerosil 200® or a crosslinked polyacrylic acid such as Carbopol 940® may be used, for example.

The gelling agents or thickeners may be chosen, for example, from polyacrylamides, acrylates, for instance Pemulen®, cellulose derivatives, for instance hydroxypropyl cellulose, or natural gums.

The moisturizers used in the compositions of the invention may be chosen, for example, from a polyol, sorbitol, maltitol, pentaerythritol, polyglyceryl acrylates and methacrylates, glycerol or glycerol derivatives. Emollients such as an alkyl malate, isohexadecane, capric or caprylic acid triglycerides, etc. may also be added.

Preserving agents that are common in the art of dermatological or cosmetological compositions may be used in the invention, for example benzoic acid and an alkyl p-hydroxybenzoate such as methyl or propyl p-hydroxybenzoate (methyl paraben and propyl paraben), or imidazolidinylurea.

The constituents of the fatty phase, i.e. the oils and lipids, may be chosen from jojoba oil, corn oil, liquid petroleum jelly, hydrogenated coconut oil, safflower oil, saturated fatty acid glycerides, stearic acid, palmitic acid, octyl stearate, glyceryl palmitate, octyl palmitate, a capric or caprylic acid triglyceride, 2-octyldodecanol, lanolin alcohol, polyethylene glycol, 2-ethylhexyl adipate, or silicone oils such as methylphenylpolysiloxane, dimethicone, cyclomethicone, cyclomethicone/dimethicone copolyol or phenyl trimethicone.

The composition may also contain, besides water (preferably demineralized water), a specific solvent such as an alcohol, for instance ethanol, or a diethylene glycol ether, for instance ethoxydiglycol or diethylene glycol monomethyl ether (Transcutol®).

Hydrophilic or lipophilic ultraviolet screening agents and sunblocks may advantageously be incorporated into the composition of the invention, and for example titanium oxides or zinc oxides, or alternatively natural sunscreens such as an extract of Pongamia, may be incorporated.

The pH of the composition is preferably between 5.5 and 7.5 and may be adjusted, depending on the compositions, by adding an acid such as citric acid or a base such as sodium hydroxide.

The solvent is chosen as a function of the components used and the administration form envisaged. It may consist of water, and preferably of demineralized water, and also a specific solvent such as propylene glycol, an alcohol, in particular ethanol, or a diethylene glycol ether.

It may also be advantageous to incorporate in the composition an additional active agent intended to improve the behavior of skin, such as tocopherol, vitamin A (retinol), retinoic acid, bactericides, etc.

In the case of gelled compositions of transparent or translucent form, for example sera, it may be advantageous to use capsules or liposomes in order to limit the amounts of emulsifier or solubilizing agent. Encapsulated forms also have the advantage of giving controlled release of the active agent over time and thus more continuous action. Depending on the size of the capsules, visual and colored effects may also be obtained.

The capsules used in the invention may comprise an envelope of hydroxyethylcellulose and may contain macadamia oil in combination with the unsaponifiable materials or the liposoluble substance (about 20%). The degree of encapsulation is generally about 10% (i.e. 0.2% unsaponifiable materials).

The composition in accordance with the present invention may be in the forms conventionally used for topical application, i.e. in the form of a gel, a lotion, an emulsion (in particular a cream or a milk), a mask, a stick or a pomade, containing common compatible and pharmaceutically acceptable excipients and supports. These topical administration forms are prepared by the known techniques, and for example, in the case of a cream, by dispersing a fatty phase in an aqueous phase to obtain an oil-in-water emulsion, or conversely to prepare a water-in-oil emulsion. In the case of creams, it is preferred to use emulsions of lamellar structure containing only a small amount of ethoxylated products or containing none at all.

By way of example, compositions in accordance with the invention may be prepared in the form of regenerating or protective creams, regenerating masks, after-sun repair creams, firming milks, antisun products or shampoos.

The examples that follow illustrate the invention in greater detail without limiting its scope. In all the composition examples that follow, the parts are expressed on a weight basis, unless otherwise indicated.

### EXAMPLE 1

An antiageing cream having the weight composition below is prepared according to the standard techniques.

| | |
|---|---|
| Unsaponifiable material from cocoa | 0.50 |
| Cocoa butter | 6.00 |
| Cocoa amino acids | 2.00 |
| Encapsulated cocoa polyphenols | 2.00 |
| Hydrogenated lecithin | 4.00 |
| Glycerol | 2.00 |
| Tetrasodium EDTA | 0.10 |
| Phenoxyethanol | 0.50 |
| Chlorophenesin | 0.10 |
| Dehydroacetic acid | 0.05 |
| Carbomer | 0.25 |
| Tromethamine | 0.20 |
| Isodecyl neopentanoate | 4.00 |
| Cetyl palmitate | 2.00 |
| Trioctyl citrate | 2.00 |
| Dimethicone | 1.00 |
| Cocoa oil | 1.00 |
| Tocopherol | 0.70 |
| Vitamin A palmitate | 0.25 |
| Moisturizer | 5.00 |
| Sunscreen | 2.00 |
| Fragrances | 0.20 |
| Water | qs 100.00 |

This composition is applied to the skin in several daily applications.

### EXAMPLE 2

An antiageing serum having the composition below is prepared.

| | |
|---|---|
| Cocoa amino acids | 2.00 |
| Encapsulated cocoa polyphenols | 2.00 |
| Soybean proteins | 10.00 |
| Butylene glycol | 3.00 |
| Glycereth 26 | 5.00 |
| Tromethamine | 0.55 |
| Pemulen TR1 (gelling agent) | 0.50 |
| Nipastat | 0.30 |
| Tween 20 | 0.40 |
| Moisturizing derivative | 5.00 |
| Refreshing agent | 0.02 |
| Glycolyzate from Terminalia sericea | 2.00 |
| Seaweed extract | 2.00 |
| Fragrances | 0.10 |
| Water | qs 100.00 |

### EXAMPLE 3

A mask containing cocoa derivatives having the composition indicated below is prepared.

| | |
|---|---|
| Cocoa amino acids | 2.00 |
| Colored capsules of unsaponifiable | |
| materials from cocoa (size 700 μm) | 10.00 |
| Encapsulated cocoa polyphenols | 2.00 |
| Soybean proteins | 10.00 |
| Macadamia oil | 1.00 |
| Sweet almond oil | 1.00 |
| Ethyl hydroxystearate | 4.00 |
| Cetostearyl alcohol | 3.00 |
| Cyclomethicone | 3.00 |
| Hydrogenated coconut glycerides | 5.00 |
| Tocopherol | 0.50 |
| Sodium acrylate/sodium acryloyl- | |
| dimethyllaurate and isohexadecane | |
| and Polysorbate 80 (Simulgel EG ®) | 3.00 |
| Mucilages from baobab | 5.00 |
| 1,3-Butylene glycol | 4.00 |
| Tetrasodium EDTA | 0.10 |
| Phenoxyethanol | 0.40 |
| para-Hydroxybenzoates mixture | 0.30 |
| Fragrances | 0.20 |
| Water | qs 100.00 |

The mask composition indicated above is prepared by the standard techniques.

According to one preferred technique, the fatty substance is melted at a temperature of about 70° C. The water and the glycols are heated to about 80° C., the preserving agents are dissolved and the mixture is cooled to 70° C. An emulsion is then formed by mixing the two phases together. The resulting mixture is allowed to cool and the polymer is added at 50° C.; the resulting mixture is then mixed for about 10 minutes and the baobab derivative, the cocoa amino acids and the capsules of unsaponifiable materials are successively added, at 40° C.

The fragrances are added to the composition at a temperature of about 35° C.

1. A cosmetic and/or dermatological composition, characterized in that it essentially comprises an extract of cocoa containing polyphenols.

2. The cosmetic and/or dermatological composition as claimed in claim 1, characterized in that it comprises an extract of cocoa containing, in combination, polyphenols and amino acids.

3. The composition as claimed in either of the preceding claims, characterized in that the extract also contains a concentrate of unsaponifiable materials.

4. The composition as claimed in claim 3, characterized in that it comprises:
from 0.05% to 5% by weight of amino acids extracted from cocoa;
from 0.05% to 5% by weight of polyphenols extracted from cocoa;
from 0.05% to 3% by weight of a concentrate of unsaponifiable materials.

5. The composition as claimed in any one of the preceding claims, characterized in that the polyphenols are encapsulated.

6. The composition as claimed in any one of the preceding claims, characterized in that it also contains a lecithin.

7. The composition as claimed in claim 6, characterized in that the lecithin is hydrogenated.

8. The composition as claimed in claim 1, characterized in that the extract is obtained from cocoa beans (Theobroma cacao).

9. The use of a composition as claimed in any one of the preceding claims, for the cosmetological treatment of the skin.

10. A cosmetological process for treating the skin, characterized in that it consists in applying to the skin a composition as claimed in any one of claims 1 to 8.
